(19) United States
(12) Patent Application Publication
(10) Pub. No.: US 2025/0009561 A1
(43) Pub. Date: Jan. 9, 2025

(54) FLEXIBLE BANDAGE WITH KINESIOLOGY TAPE

(71) Applicant: McKenna Haase, Greenwood, IN (US)

(72) Inventor: McKenna Haase, Greenwood, IN (US)

(21) Appl. No.: 18/761,683

(22) Filed: Jul. 2, 2024

Related U.S. Application Data
(60) Provisional application No. 63/511,737, filed on Jul. 3, 2023.

(51) Int. Cl. A61F 13/0203; A61F 13/00; A61F 13/02; A61L 15/18; A61L 15/24; A61L 15/40

(52) U.S. Cl. CPC A61F 13/022; A61F 13/00076; A61F 13/0269; A61L 15/18; A61L 15/24; A61L 15/40

(57) ABSTRACT

A multi-sized bandage capable of withstanding harsher work or use environments provides needed support for injuries, such as wounds. The bandage is sized larger and includes layers to increase the integrity of the product to allow for the harsh conditions, while providing a cleaner dressing for wounds. The bandage includes a wound dressing, bandage layer, and a kinesiology tape layer to be able to hold the bandage in place on a body part. The incorporation of the layers reduces the likelihood of the bandage moving or even falling off the user during continued work in different environments.

### CROSS REFERENCE TO RELATED APPLICATIONS

This application claims priority under 35 U.S.C. § 119 to provisional patent application U.S. Ser. No. 63/511,737, filed Jul. 3, 2023. The provisional patent application is herein incorporated by reference in its entirety, including without limitation, the specification, claims, and abstract, as well as any figures, tables, appendices, or drawings thereof.

### TECHNICAL FIELD

The present disclosure relates generally to an apparatus having applications in at least the medical industries, such as for the treatment of wounds. More particularly, but not exclusively, the present disclosure relates to improved bandages that are able to withstand harsh conditions.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

A bandage is a piece of material used either to support a medical device such as a dressing or splint, or on its own to provide support to or to restrict the movement of a part of the body. When used with a dressing, the dressing is applied directly on a wound, and a bandage used to hold the dressing in place. Other bandages are used without dressings, such as elastic bandages that are used to reduce swelling or provide support to a sprained ankle. Tight bandages can be used to slow blood flow to an extremity, such as when a leg or arm is bleeding heavily. Some bandages include an adhesive to aid in holding the bandage and dressing in place on the body, while others can be adhered or otherwise affixed in other manners.

Bandages are available in a wide range of types, from generic cloth strips to specialized shaped bandages designed for a specific limb or part of the body.

Within reason, the bandage has not changed extensively in approximately 100 years (since 1921 to be exact). With excessive wrapping, flimsy materials, and too small of sizing, many individuals decide to go without. Perhaps more popular, many people who work with their hands prefer paper towels and duct tape over the traditional bandage.

While surgical bandages are designed for medical settings, due to the inconvenience and lack of quality for working hands, oftentimes these bandages create less sanitation for working and athletic settings. Because construction workers, mechanics, athletes, etc. want to get back on the job after a cut or oozing blister, the choice to skip the flimsy, traditional bandage often leads to contamination and biohazards.

Therefore, there is a need in the art for an accessible, durable bandage, properly sized and built to last that can be easily stripped and placed on the job so doers can get back to what they do best—doing.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of any of the aspects and/or embodiments of the present disclosure to improve on or overcome the deficiencies in the art.

It is a further object, feature, and/or advantage of any of the aspects and/or embodiments of the present disclosure to include an athletic style bandage for treating wounds, such as on the hands, fingers, arms, or legs of a user.

It is still yet a further object, feature, and/or advantage of any of the aspects and/or embodiments of the present disclosure to provide an improved bandage that is easy to access and apply.

It is still yet a further object, feature, and/or advantage of any of the aspects and/or embodiments of the present disclosure to package improved bandages in a manner where they do not need to be individually wrapped for easy access and less chance of contamination prior to application.

The bandages disclosed herein can be used in a wide variety of applications. For example, as noted, they can be fitted for generally any part of the body and able to withstand harsh conditions while staying adhered to a person.

It is preferred that the apparatus be safe, cost effective, and durable.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the apparatus.

Methods can be practiced which facilitate use, manufacture, assembly, maintenance, and repair of an apparatus which accomplish some or all of the previously stated objectives.

The bandages can be incorporated into systems or kits which accomplish some or all of the previously stated objectives.

According to at least some aspects of the present disclosure, a bandage comprises a first layer comprising an adhesive on at least one side; a non-adherent pad comprising a wound dressing on the adhesive side of the first layer; and a second layer positioned on the first layer opposite the wound dressing, the second layer comprising kinesiology tape.

According to at least some aspects of some embodiments of the present disclosure, the second layer comprises an acrylic wave pattern adhesive to adhere the second layer to the first layer.

According to at least some aspects of some embodiments of the present disclosure, the second layer comprises an elastic cotton strip.

According to at least some aspects of some embodiments of the present disclosure, the wound dressing comprises an absorbent material.

According to at least some aspects of some embodiments of the present disclosure, the first layer comprises a hypoallergenic material.

According to at least some aspects of some embodiments of the present disclosure, the first layer comprises a zinc oxide infused cotton substrate.

According to at least some aspects of some embodiments of the present disclosure, the wound dressing is sized less than the size of the first layer.

According to at least some aspects of some embodiments of the present disclosure, the second layer comprises a cotton strip with an acrylic wave pattern adhesive.

According to at least some aspects of some embodiments of the present disclosure, the bandage further comprises at least one protective paper layer covering the non-adherent pad and the adhesive side of the first layer.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

[The patent or application file contains at least one drawing executed in color. Copies of this patent or patent application publication with color drawing(s) will be provided by the Office upon request and payment of the necessary fee.]

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a view of a bandage according to at least some aspects of the present disclosure.

FIG. 2 is a view of a wave pattern of adhesives used with a layer of the bandage of FIG. 1.

FIG. 3 is one size of many of the variations of a bandage as disclosed herein.

FIG. 4 is another size of many of the variations of a bandage as disclosed herein.

FIG. 5 is another size of many of the variations of a bandage as disclosed herein.

FIG. 6 is another size of many of the variations of a bandage as disclosed herein.

FIG. 7 is a depiction of a variation of a bandage as disclosed herein.

FIG. 8 is a depiction of a variation of a bandage as disclosed herein.

FIG. 9 is a depiction of a variation of a bandage as disclosed herein.

FIG. 10 is a depiction of a variation of a bandage as disclosed herein.

FIG. 11 is a depiction of a variation of a bandage as disclosed herein.

FIG. 12 is a depiction of a variation of a bandage as disclosed herein.

FIG. 13 is a depiction of a variation of a bandage as disclosed herein.

FIG. 14 is a depiction of a variation of a bandage as disclosed herein.

FIG. 15 is a depiction of a variation of a bandage as disclosed herein.

FIG. 16 is a depiction of a variation of a bandage as disclosed herein.

FIG. 17 is a depiction of a variation of a bandage as disclosed herein.

FIG. 18 is a depiction of a variation of a bandage as disclosed herein.

FIG. 19 is a depiction of a variation of a bandage as disclosed herein.

FIG. 20 is a depiction of a variation of a bandage and packaging as disclosed herein.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Referring now to the figures, an improved bandage 10 is shown. As will be appreciated, the bandage 10 of the present disclosure includes improvements over standard adhesive bandages, such as the ability to withstand harsher environments of use, which will allow for greater usage and an increase in the cleanliness of wounds incurred by users of the improved bandage 10. While the bandage may be referred to as an athletic style bandage, it should be appreciated that it can be used by people in any number of industries, including, but not limited to mechanics, farmers, construction workers, athletes, and individuals who work in tough or physical environments, often working with their hands. As will be understood, the bandage 10 of the present disclosure provides an accessible, durable bandage, properly sized and built to last that can be easily stripped and placed on the job so doers can get back to what they do best—doing.

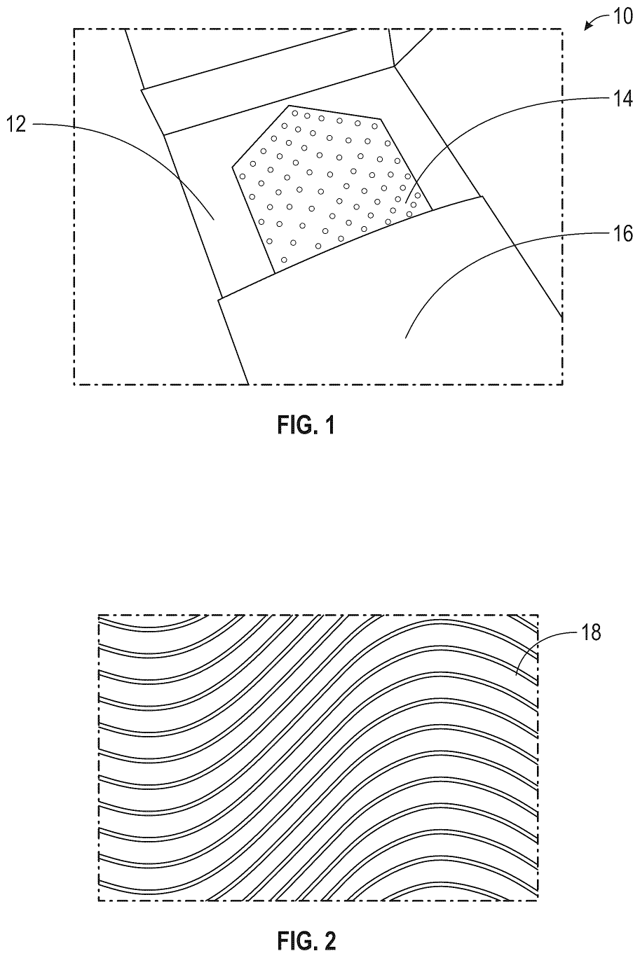

Therefore, in order to provide such a more rugged, yet clean bandage, aspects and/or embodiments of the present disclosure provide an improved bandage 10. One example of such a bandage 10 is shown in FIG. 1. The bandage 10 includes a first layer 12, which comprises an adhesive on at least one side. The adhesive may be any adhesive to aid in holding the bandage 10 in place, such as on or around a portion of a body part of a user. Thus, the bandage 10 can be referred to as an adhesive bandage, at least according to some embodiments.

The adhesive bandage protects the wound and scab from friction, bacteria, damage, and dirt. Thus, the healing process of the body is less disturbed. An adhesive bandage is a small, flexible sheet of material which is sticky on one side. The adhesive sheet is usually a woven fabric, plastic (PVC, polyethylene, or polyurethane), or latex strip. It may or may not be waterproof; if it is airtight, the bandage is an occlusive dressing. The adhesive is commonly an acrylate, including methacrylates and epoxy diacrylates (which are also known as vinyl resins).

Positioned on the first layer 12 (adhesive side) of the bandage 10 is a dressing portion 14, which may be referred to as an absorbent pad. The absorbent pad is often made of cotton, and there is sometimes a thin, porous-polymer coating over the pad, to keep it from sticking to the wound. The pad may also be medicated with an antiseptic solution. In some bandages, the pad is made of a water-absorbing hydrogel. This is especially common in dressings used on blisters, as the gel acts as a cushion.

Also shown covering a portion of the adhesive first layer 12 and the wound dressing pad 14 is protective paper 16, which may also be known as backing paper. The backing paper mitigates the adhesive bandage 10 from sticking to another bandage during packaging, mitigates sticking to unwanted surfaces, and also mitigates contamination of the bandage prior to use. According to at least some aspects of some embodiments, the backing paper. The backing and bag are often made of coated paper but may be made of plastic.

Thus far, the bandage has been shown to include similar layers as most adhesive bandages. However, in order to increase the durability of the bandage to be used in harsher or more rugged environments, another layer is added. The additional layer may be known as a second layer 18 and is located opposite the wound dressing 14 on the first layer 12. This provides the additional support for the bandage 10.

The second layer 18 comprises kinesiology tape that is adhered to the first layer 12. This layer further comprises an elastic cotton strip with an acrylic wave pattern (see, e.g., FIG. 2) adhesive that is flexible, durable, and sized larger than most common adhesive bandages used today.

Elastic therapeutic tape, also called kinesiology tape or kinesiology therapeutic tape, Kinesio tape, k-tape, or KT is an elastic cotton strip with an acrylic adhesive that is purported to ease pain and disability from athletic injuries and a variety of other physical disorders. The k-tape is a type of thin, elastic cotton tape that can stretch up to 140% of its original length. As a result, if the tape is applied stretched greater than its normal length, it will “recoil” after being applied and therefore create a pulling force on the surface adhered thereto. This elastic property allows much greater range of motion compared to traditional white athletic tape and can also be left on for long periods before reapplication.

In addition, while K-tape has been called out, it should be noted that generally any material for a base that that is similar to or otherwise includes desired properties could be used as well. It should be noted that alternatives could be used instead of or in addition to this layer. For example, a layer could comprise a gentle cotton fiber blend, medical grade acrylic adhesive, latex-free, hypoallergenic, and also be water-resistant.

It should also be appreciated that traditional athletic tape be used. Such a tape comprises non elastic adhesive athletic tapes usually come in cotton and/or polyester and have a zinc oxide adhesive mass backing.

Designed to mimic human skin, with roughly the same thickness and elastic properties, the tape can be stretched 30-40% longitudinally. It is a latex-free material with acrylic adhesive, which is heat activated. The cotton fibers allow for evaporation and quicker drying leading to longer wear time, up to 4 days.

Therefore, the combination of layers of the bandage 10 as included herein provide advantages over the commonly used adhesive bandage, which does not hold up to the harsh environments as described herein. Still further, the design will allow for easier application so that wounds are covered in a manner to mitigate exposure to unwanted elements, such as dirt and bacteria.

For example, as will be understood, the bandages 10 can take numerous sizes and shapes. For ease of access and attachment, the bandages can be stacked in packaging relative to one another without needing to be individually packaged, as is the case with standard adhesive bandages. This is due, in part, to the paper backing that covers the adhesive components of the bandage 10, which mitigates the bandages from sticking to one another. The bandages 10 can be stored in a housing in a stacked manner, whereas one hand can grab a bandage and remove the paper backing before applying the bandage to the wound or body part with ease.

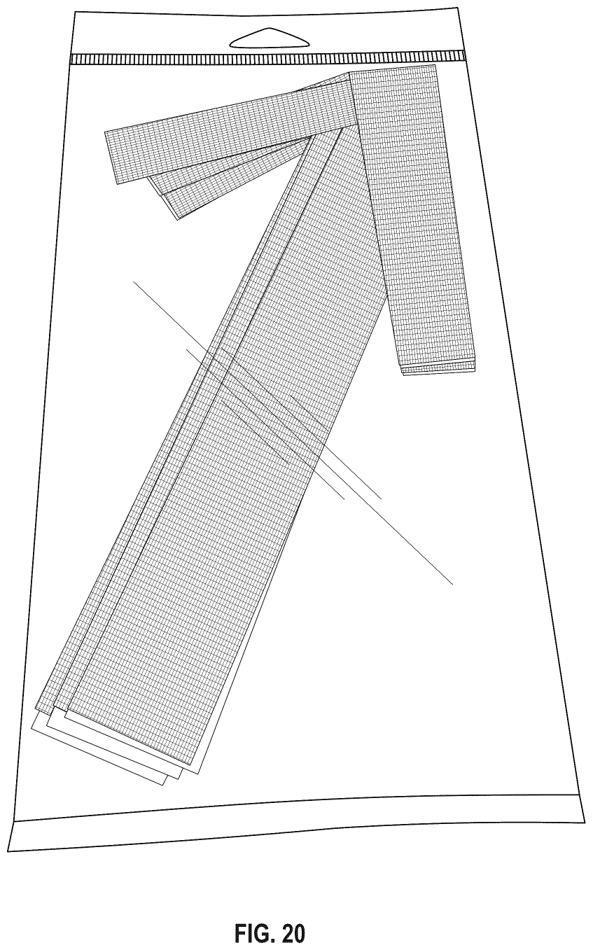

The package can be set up such that the bandages lie flat similar to a pack of stickers, which reduces the potential of the bandages sticking to one another in the package housing. Example packaging is included and shown in the depiction of FIG. 20. As shown, the package can be clear, opaque, or partially opaque. The packaging includes multiple bandages housed together. This can be all of the same style of bandages or different sizes and styles of bandages in a common package. As shown, the bandages are simply arranged in a stacked manner that does not include individual packaging for each bandage. Once opened, the paper backing will protect the bandages and mitigate them sticking to one another. However, when needed, the bandages will be easily accessible, especially if one hand or appendage is limited, for example due to a wound thereon or thereat.

However, it should be appreciated that this is only an example of packaging, and that other or additional packaging could be used. This could include tins, boxes, bags, or generally any other device that could hold one or more bandages for future use. According to some aspects, it may be desirable that the packaging be resealable or closeable, such as to mitigate any unwanted contamination of the bandages prior to use. This could be by way of a closeable or resealable packaging. For example, a package similar to that shown in FIG. 20 could include a layer of resealable adhesive. This would allow the interior of the package to be easily accessible for a user but would provide a barrier portion to limit or even prevent contamination of the bandages that have not yet been used. However, it should be appreciated that the exact type and/or size of packaging need not be limiting on the present disclosure.

Still further, it should be noted that some packaging come with additional objects, such as creams, sprays, gels, or the like, which can aid in the care of a wound. This can include antibiotics, antiseptic gels, sprays, glues, or other treatments for wounds that could be used in conjunction with the bandages.

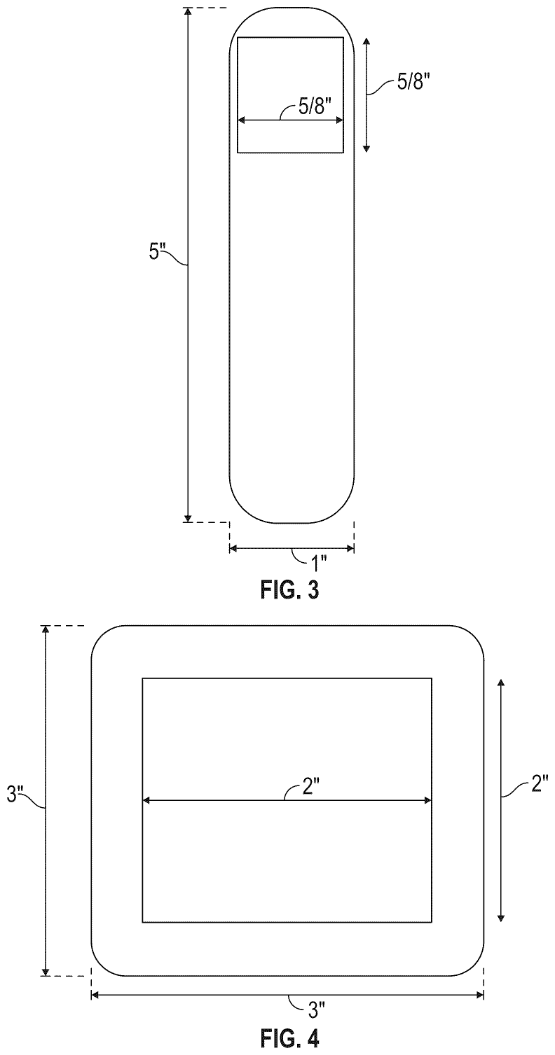

FIGS. 3-6 disclose examples of sizes and shapes of bandages including the properties as disclosed herein. It should be noted that the shapes and/or sizes are not limiting and are included for illustrative purposed.

FIG. 3 discloses a standard shaped bandage 10 that includes a wound dressing near one end (shown by the white box in the otherwise black depiction of the bandage). The positioning of the dressing 14 near an end instead of in the middle provides advantages. For example, the wound dressing can be positioned on the wound and the remaining body portion of the adhesive bandage 10 wrapped around the body portion with one hand. This is especially easy for attaching to a finger when one has to apply the bandage themselves. The size of the bandage can be varied, such as to wrap about a larger body portion (wrist or arm) as well.

FIG. 4 is a larger bandage 10 that can be applied to cover a larger wound. Again, the white portion depicts a wound dressing portion, with the black portion comprising the adhesive portion. The portions are sized to mitigate removal of the bandage once placed to cover the wound.

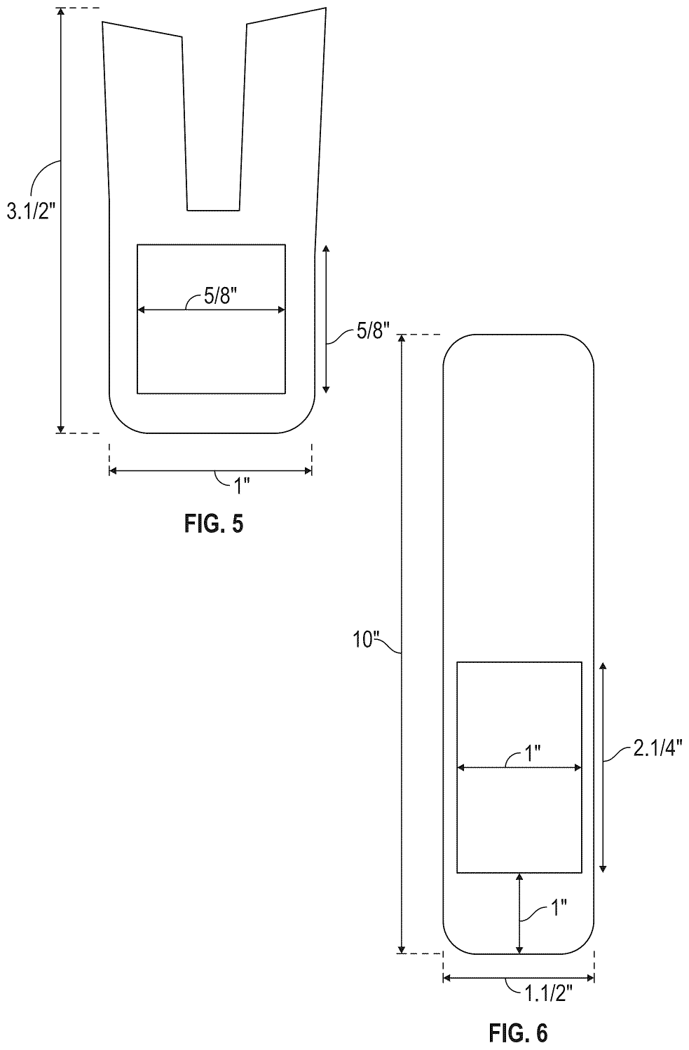

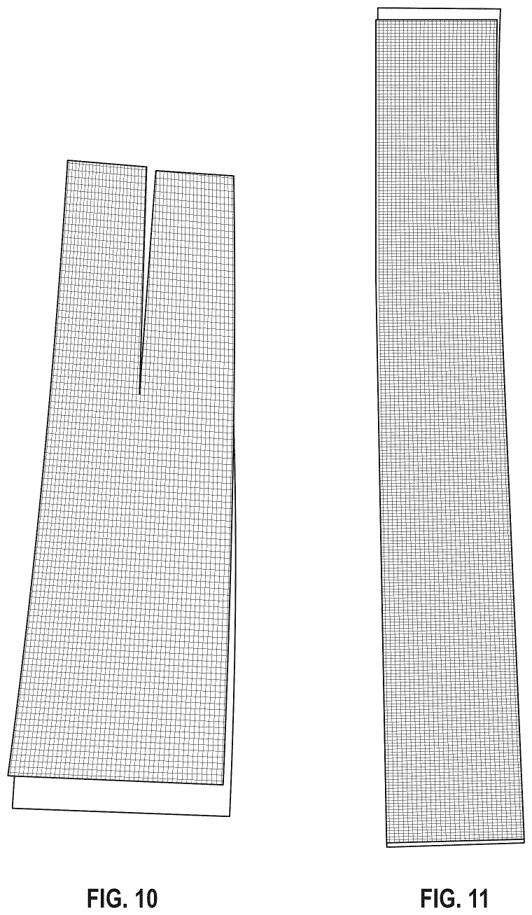

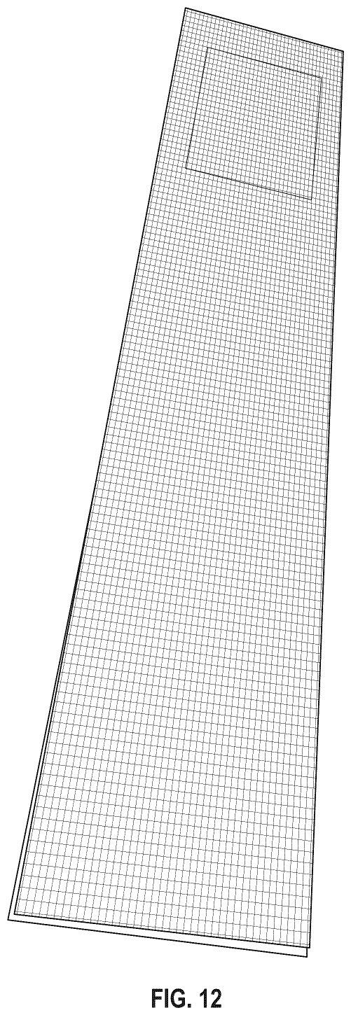

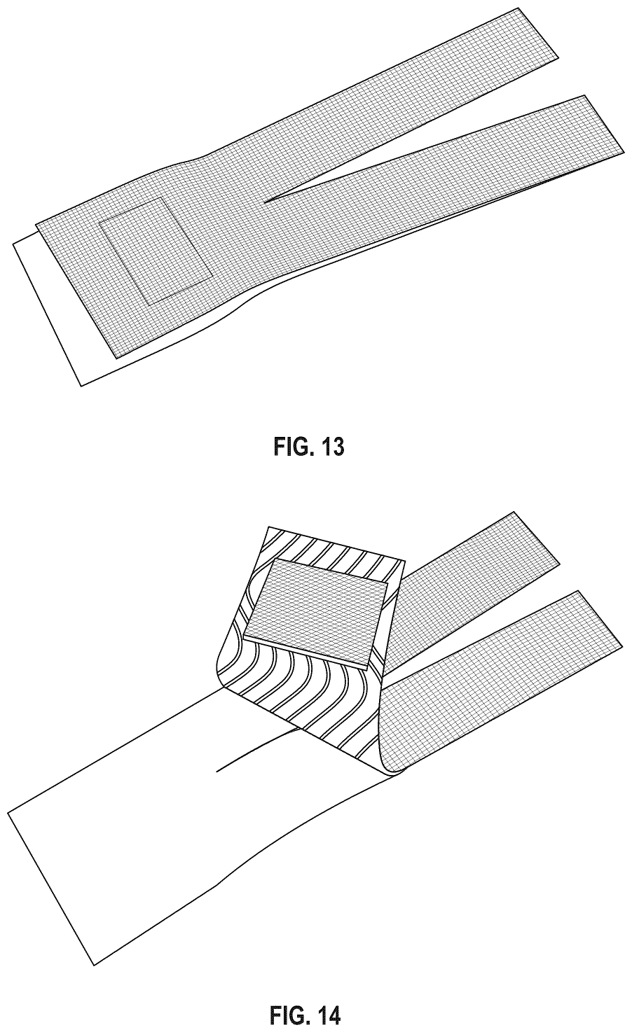

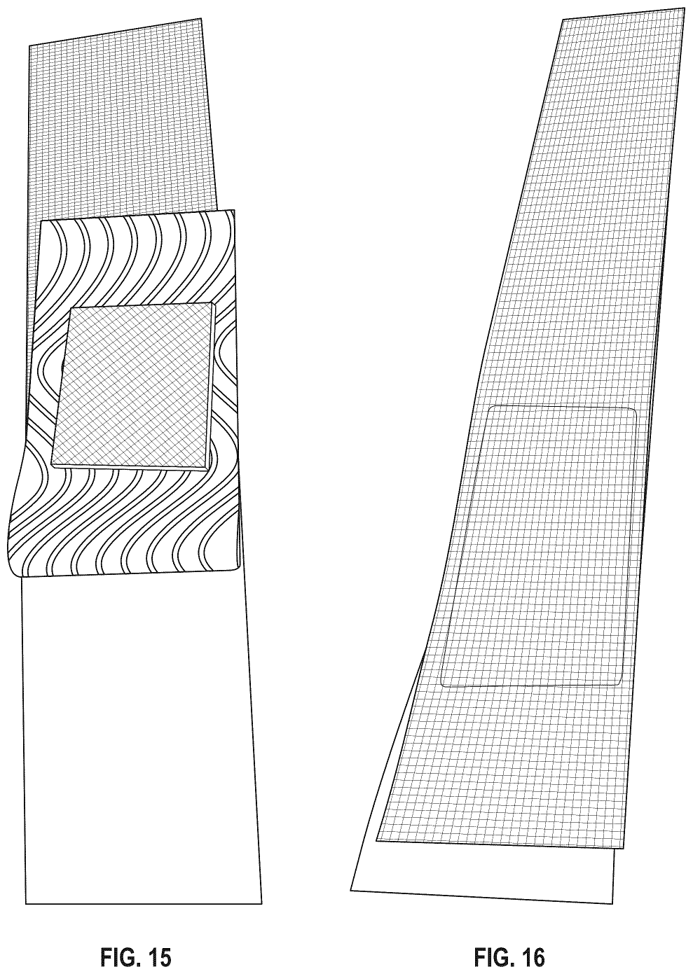

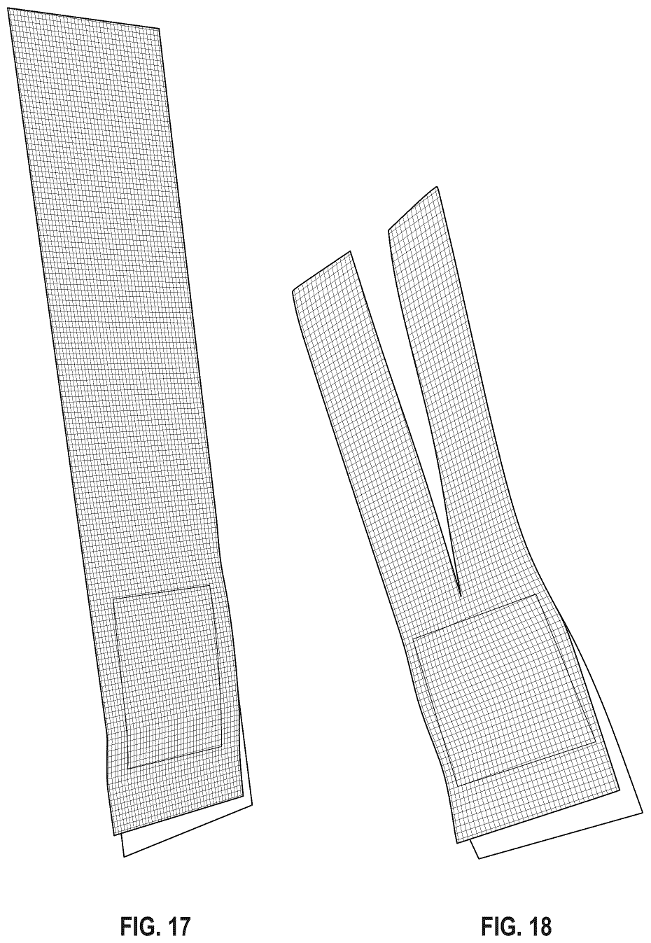

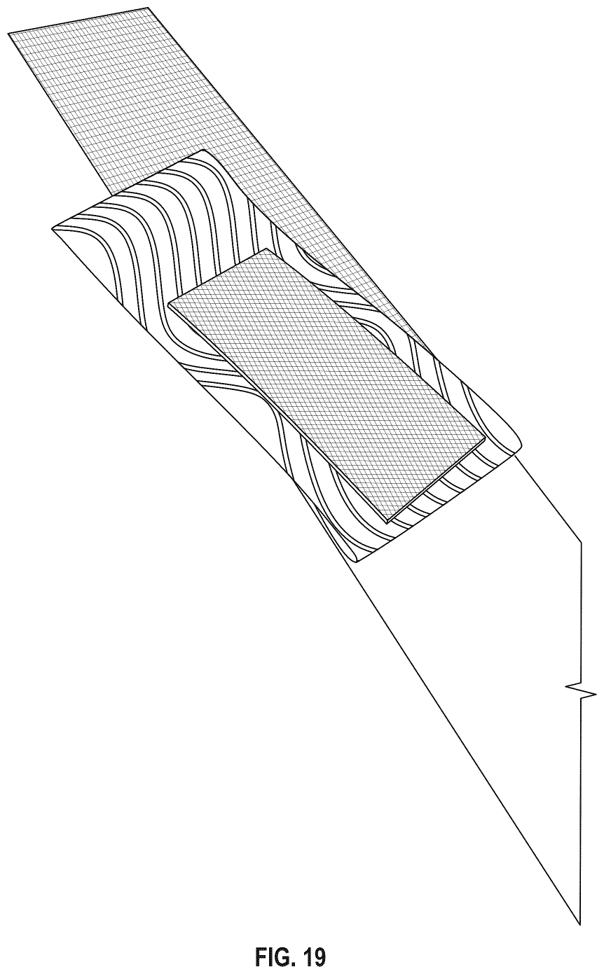

FIG. 5 depicts an irregularly shaped bandage with a wound dressing again depicted. The adhesive portion (black portion) includes a cutout and two leg-like projections extending from the wound dressing portion. This allows the adhesive to be placed between figures or otherwise wrapped about a user to best hold the bandage in place to cover and protect the wound. Additional versions or similar versions of a bandage as shown in FIG. 5 are included in FIGS. 10, 13, 14, and 18. These are depictions that show the location of the wound dressing and also more details on the layers of the bandage.

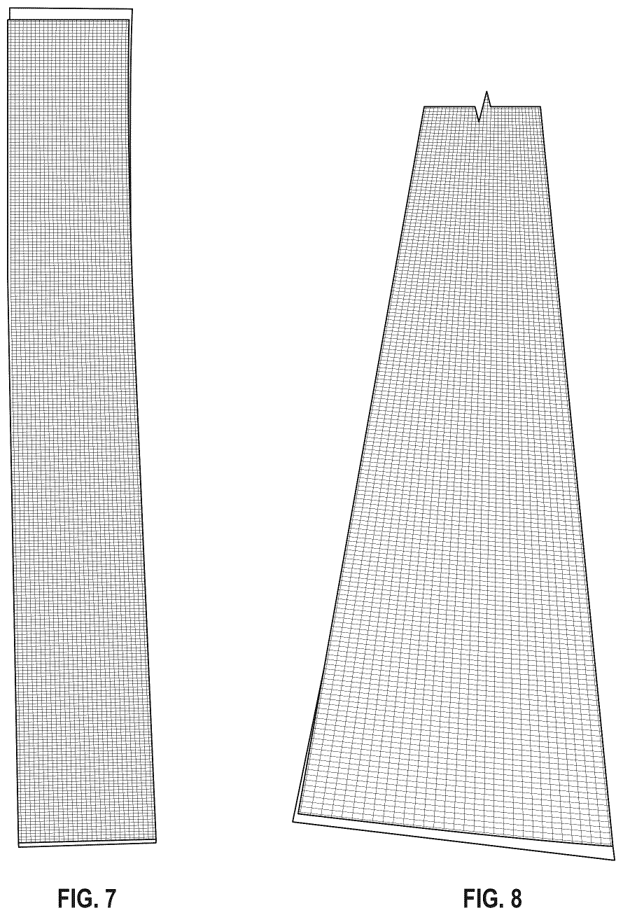

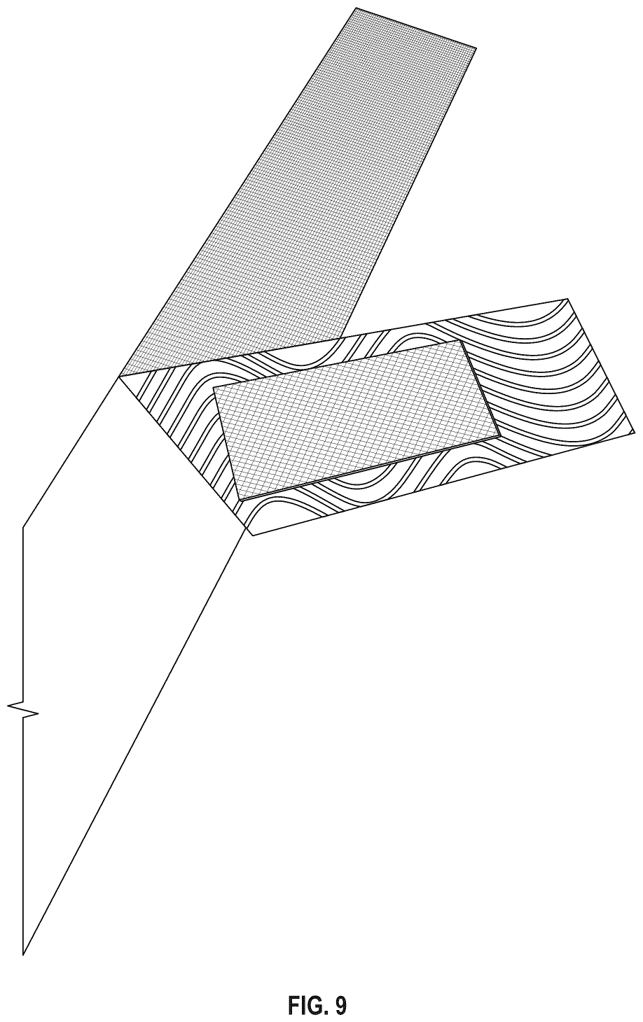

FIG. 6 is similar to FIG. 3 but is larger in size. As noted, such a shaped bandage could be used for body parts including, but not limited to arms or legs. The longer length of the adhesive portion allows for a greater part of the adhesive to be attached to the user's body, which will mitigate the bandage from becoming detached from the user. This can also wrap around a hand. A similar version of such a bandage is shown in the depiction in FIGS. 7-9. The multiple layers are shown in the figures. This includes, for example, the paper backing being removed to show the wound dressing layer, which is sized smaller than the rest of the layers. This allows the bandage to be wrapped around a portion of the user while protecting a wound.

Additional aspects and/or features of a bandage covered herein are to be considered a part of the disclosure. For example, the bandage could be hypoallergenic.

Again, note that these are not the only shapes and/or sizes of bandages, and any shape or size should be considered a part of the disclosure. Therefore, the bandages as disclosed herein can be used for harsh conditions. For example, farm workers, athletes, mechanics, fabricators, blue collar workers, construction workers, firefighters, police, medical workers, and any harsh working environment can benefit from the disclosed bandage.

It should be appreciated that variations and/or changes to any of the components or embodiments that are obvious to those skilled in the art are to be considered a part of the present disclosure. In addition, any of the aspects of any of the embodiments disclosed could be combined in ways not explicitly shown and/or described to provide yet additional embodiments that are part of the disclosure. The disclosure is not to be limited to the embodiments disclosed herein.

1. A bandage, comprising:
a first layer comprising an adhesive on at least one side;
a non-adherent pad comprising a wound dressing on the adhesive side of the first layer; and
a second layer positioned on the first layer opposite the wound dressing, the second layer comprising kinesiology tape.

2. The bandage of claim 1, wherein the second layer comprises an acrylic wave pattern adhesive to adhere the second layer to the first layer.

3. The bandage of claim 1, wherein the second layer comprises an elastic cotton strip.

4. The bandage of claim 1, wherein the wound dressing comprises an absorbent material.

5. The bandage of claim 1, wherein the first layer comprises a hypoallergenic material.

6. The bandage of claim 1, wherein the first layer comprises a zinc oxide infused cotton substrate.

7. The bandage of claim 1, wherein the wound dressing is sized less than the size of the first layer.

8. The bandage of claim 1, wherein the second layer comprises a cotton strip with an acrylic wave pattern adhesive.

9. The bandage of claim 1, further comprising at least one protective paper layer covering the non-adherent pad and the adhesive side of the first layer.

10. A bandage, comprising:
a first layer comprising an adhesive on at least one side; and
a second layer positioned on the first layer opposite the wound dressing, the second layer comprising kinesiology tape, wherein the second layer comprises an acrylic wave pattern adhesive to adhere the second layer to the first layer.

11. The bandage of claim 10, further comprising a non-adherent pad comprising a wound dressing on the adhesive side of the first layer.

12. The bandage of claim 11, wherein the wound dressing comprises an absorbent material.

13. The bandage of claim 10, wherein the first layer comprises a hypoallergenic material.

14. The bandage of claim 10, wherein the first layer comprises a zinc oxide infused cotton substrate.

15. The bandage of claim 10, wherein the second layer comprises an elastic cotton strip.

16. The bandage of claim 10, further comprising at least one protective paper layer covering the non-adherent pad and the adhesive side of the first layer.

17. A bandage system, comprising:
a bandage comprising a first layer comprising an adhesive on at least one side, a non-adherent pad comprising a wound dressing on the adhesive side of the first layer, and a second layer positioned on the first layer opposite the wound dressing, the second layer comprising kinesiology tape; and
a package associated with the bandage wherein one or more bandages are positioned in the package.

18. The bandage system of claim 17, wherein the package is clear, opaque, or partially opaque.

19. The bandage system of claim 17, wherein the package is resealable.

20. The bandage system of claim 17, wherein the wound dressing of the bandage comprises an absorbent material.
